(19) United States
(12) Patent Application Publication
(10) Pub. No.: US 2015/0282772 A1
(43) Pub. Date: Oct. 8, 2015

(54) PROTECTIVE ENVELOPE FOR DIGITAL DENTAL SENSOR

(71) Applicant: FLOW DENTAL PRODUCTS, LLC, Deer Park, NY (US)

(72) Inventor: William Winters, New Rochelle, NY (US)

(21) Appl. No.: 14/245,524

(22) Filed: Apr. 4, 2014

(51) Int. Cl. A61B 6/00; A61B 6/14

(52) U.S. Cl. CPC A61B 6/425; A61B 6/145

(57) ABSTRACT

A protective envelope for a digital sensor used for capturing and recording dental X-ray images when the envelope is disposed in the mouth of a person or an animal is presented. The envelope is formed with a cavity housing the sensor. The envelope further includes an external surface wrapped around the digital sensor and held in place by a tab with an adhesive surface.

### RELATED APPLICATIONS

This application is related to design application, Ser. No. ______, entitled ENVELOP FOR DIGITAL SENSORS, filed on even date herewith, ______now incorporated herein by reference.

### BACKGROUND OF THE INVENTION

A. Field of Invention

This invention pertains to an envelope for holding a digital sensor used to capture an X-ray image of a person's teeth and the like. The envelope slides over the sensor and is held in place during the capturing process.

B. Description of the Prior Art

Analog X-ray photography has been used for many years by dentists for capturing images of patients' teeth. This technology involved positioning a sheet of X-ray sensitive film behind one or more teeth and then directing an X-ray beam at the teeth of a patient. Some of the tissues and other portions of the teeth absorb the X-rays more than others, and, as a result, a latent image on the film is created that is illustrative of the internal structures of the teeth. The film is then developed using conventional chemicals thereby rendering the latent image clearly visible on the film. Of course the film can be used only once and has to be scanned, printed or otherwise processed to obtain copies for the patient's record, to share the images with others, etc.

Recently, digital X-ray photography has been replacing this analog technology. In this newer technology, the film is replaced by a flat digital sensor that includes a digital sensor array. The digital sensor is placed in the mouth, behind the teeth and is used to record a digital image of the teeth directly when the sensor is exposed to X-rays. An advantage of this technology is that the image is obtained quickly and this image can be copied and shared at will without the need for any chemical processing.

Another advantage is that the sensor can be reused easily. However, since the sensor is placed into the mouths of several persons, it must be kept sterile. Typically this is accomplished by placing the sensor into a thin plastic envelope.

Digital sensors come in various sizes depending, for example, on the manufacturers and, accordingly, the envelopes also come in several sizes to accommodate the sensors. A problem with digital sensors and the corresponding envelops for holding the sensors has been that the envelopes cannot be made to fit the sensors very snugly otherwise the sensors are difficult to remove from the envelopes. As a result, a sensor disposed inside the envelope tends to move around and even fall out. If the sensor moves around while in the mouth, a fuzzy inaccurate digital image may be created. This problem is especially pronounced if a inadvertently, a person uses an envelope that is a little large for the sensor because it was meant for another sensor.

Moreover, it is well known that ideally a front face of the digital sensor must be aligned in parallel to the vertical place defined by the teeth, otherwise the image captured by the sensor will be skewed. However, if the envelope is wider than the sensor, the sensor has tendency to tilt backward so that the front face of the sensor is not aligned properly .e.g, in parallel with the vertical plane of the teeth. The present invention solves these problems.

### SUMMARY OF THE INVENTION

Briefly, the present invention pertains to a protective envelope for housing a digital sensor while the digital sensor is being exposed to radiation as a patient is holding the digital sensor in the mouth. The protective envelope includes an envelope body having a top surface and a bottom panel defining a cavity therebetween. The cavity has an opening and is sized and shaped to receive the digital sensor through said opening for recording dental X-rays as the envelope is positioned within the mouth. A tab is formed on the envelope body and has an adhesive surface. The envelope body and tab are constructed and arranged for wrapping around the digital sensor inside the cavity, said envelope body being maintained in the wrapped position by said tab.

### BRIEF DESCRIPTION OF THE DRAWINGS

FIG. 1 shows a plan view of a protective envelope constructed in accordance with this invention;

FIG. 2 shows a bottom view of the envelope of FIG. 1;

FIG. 3 is an elevational cross-sectional view of the envelope of FIGS. 1 and 2;

FIG. 4 shows an orthogonal view of the envelope of FIGS. 1-3;

FIG. 5 shows a cross-sectional view of the double sided adhesive pad for the envelope;

FIG. 6 shows a top view of the envelope wrapped around a digital sensor in accordance with this invention; and

FIG. 7 is a bottom enlarged view of the envelope of FIG. 6.

### DETAILED DESCRIPTION OF THE INVENTION

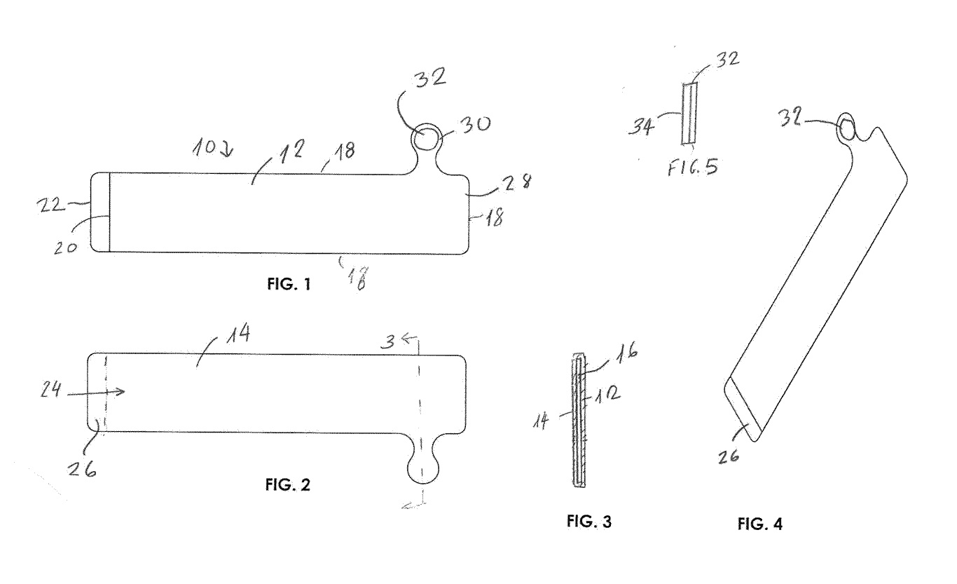

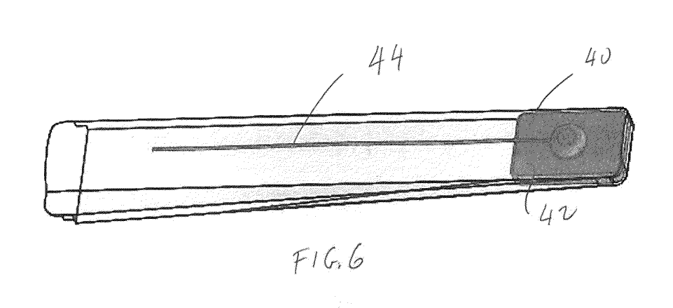

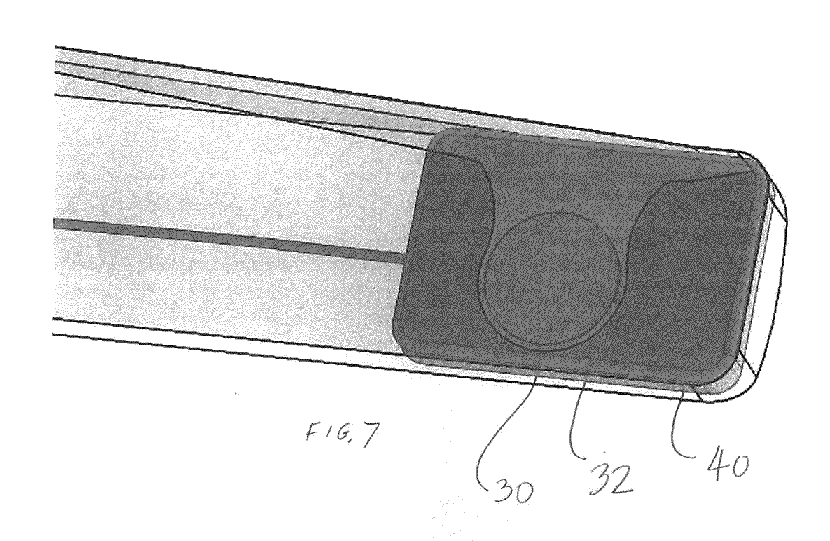

Referring first to FIGS. 1-5, the present invention pertains to an envelope 10 made of a clear plastic material. Typically the material may be about 0.5 mil thick, transparent and is very supple. The envelope 10 is formed of a front panel 12 and a back panel 14 joined along edges three of the edges 18 to form a cavity 16. The panels are joined by heat, sonic welding or other well-known means. (In FIG. 3, the thickness envelope is not shown to scale but is shown much larger for the sake of clarity).

Panels 10, 12 each has a free edge 20, 22 respectively that are not joined and therefore form a mouth 24 for accessing the cavity 16. Preferably one of the panels, for example 14 is longer than the other to form a narrow flap 26. The flap 26 is provided to facilitate separating the two panels 12, 14 and inserting the digital sensor into the cavity 16.

The panels 12, 14 are formed with a lateral tab 30 joined to one of the edges of the panels near closed end 28.

Attached to one of the surfaces of the tab 30 is a double sided adhesive pad 32. One side of the adhesive pad 32 is attached to the tab 30. The other side is pad 32 is covered with a release sheet 34.

As illustrated in FIGS. 6 and 7, a typical sensor 40 used for capturing X-ray images includes a head having a generally rectangular shape and a cable 44 extending away from the head 42 to a device (not shown) used to control the operation of the sensor 40. It should be understood that the present invention is very flexible in that it can accommodate a large variety of sensors having different shapes and sizes. Accordingly sensor 40 may have other configurations as well.

The envelope 10 is used as follows. First, the two panels 12, 14 are separated to open cavity 16 and the sensor 40 is inserted into the cavity with the head 42 being positioned as close as possible near end 28 of the envelope 10. Next, the release sheet 34 is removed from the double sided pad 32 and the tab 30 is wrapped around the head 42. The envelope 10 is sized and shaped so that it is somewhat larger widthwise than the head 42 and hence, as the tab 30 is wrapped around the head 42, some portions of the panels 12, 14 overlap and are wrapped around the head 42 as well, as can be seen in FIG. 7 with pad 32 engaging an outer surface of panel 14,. In this manner, the sensor 40 is held snugly within the envelope 10 and does not move around as it is being inserted into the mouth of a patient and exposed to an X-ray beam. It should be appreciated that because of its structure, the envelope is readily useable for sensors of various shapes and sizes without modifications. In addition to human patients, the envelope may be used for veterinary environments as well.

Moreover, since the envelope is tightly wrapped about the body of the sensor, the sensor does not have the tendency to tilt away from a parallel position with respect to the vertical plane o the teeth.

Numerous modifications may be made to this invention without departing from its scope as defined in the appended claims.

1. A protective envelope for housing a digital sensor while the digital sensor is being exposed to radiation as a patient is holding the digital sensor in the mouth, said protective envelope comprising:
an envelope body having a top surface and a bottom panel defining a cavity therebetween, said cavity having an opening and being sized and shaped to receive the digital sensor through said opening for recording dental X-rays as the envelope is positioned within the mouth; and
a tab formed on said envelope body and having an adhesive surface, said envelope body and tab being constructed and arranged for wrapping around the digital sensor inside the cavity, said envelope body being maintained in the wrapped position by said tab.

2. The protective envelope of claim 1 wherein said tab includes a first and a second surface with one of said first and said surfaces including said adhesive surface.

3. The protective envelope of claim 1 wherein said panels are joined along edges, and wherein said tab is an extension of one of said edges.

4. The protective envelope of claim 1 wherein said envelope body includes two longitudinal edges and a lateral edge, said lateral edge closing said cavity to form an end opposite said opening, said tab being disposed at a tab position closer to said end then to said opening.

5. The protective envelope of claim 4 wherein the digital sensor has sensor width and said panels have panel widths larger than said sensor width.

6. A digital sensor assembly for recording dental X-rays when the assembly is disposed in the mouth of a person or animal, said digital sensor assembly comprising:
a digital sensor;
a protective envelope having an envelope surface shaped to form a cavity with said digital sensor inside said cavity; and
a closing tab arranged and constructed to hold said protective envelope in a wrapped configuration around said digital sensor with portions of said envelope surface being overlapped on itself to keel said digital sensor tight and snug.

7. The digital sensor assembly of claim 6 wherein said tab has a flat tab body with two opposed tab surfaces, at least one of said tab surfaces having an adhesive for securing said envelope surface in said wrapped configuration.

8. The digital sensor assembly of claim 6 wherein said envelope is a plastic material.

9. The digital sensor assembly of claim 6 wherein said envelope is transparent.
